(19) United States
(12) Patent Application Publication
(10) Pub. No.: US 2026/0081021 A1
(43) Pub. Date: Mar. 19, 2026

(54) INTEGRATION OF ELECTROPHYSIOLOGICAL PROCEDURE SYSTEMS

(71) Applicant: c/o The Vektor Group, Inc., San Diego, CA (US)

(72) Inventors: Christopher J. T. Villongco, San Diego, CA (US); Robert Joseph Krummen, Bellevue, WA (US); Joseph Thomas Braidwood, Bellevue, WA (US); Christian David Marton, San Diego, CA (US); Maurice J. Pirio, Seattle, WA (US)

(21) Appl. No.: 19/110,064

(22) PCT Filed: Sep. 11, 2023
(86) PCT No.: PCT/US23/73889

§ 371 Date: Mar. 8, 2025

Related U.S. Application Data
(60) Provisional application No. 63/375,173, filed on Sep. 9, 2022.
(60) Provisional application No. 63/495,475, filed on Apr. 11, 2023.

(51) Int. Cl. G16H 50/20; G06T 7/00; G06T 17/20; G16H 10/60; G16H 20/30; G16H 50/30; G16H 50/50

(52) U.S. Cl. CPC G16H 50/20; G06T 7/0012; G06T 17/20; G16H 10/60; G16H 20/30; G16H 50/30; G16H 50/50; G06T 2207/30048

(57) ABSTRACT

Techniques are provided that support communications between electrophysiological procedure (EPP) systems during an EPP based on an EPP application programming interface (API). The EPP API specifies parameters and behavior of pull functions and push functions that support exchanging data between EPP systems during an EPP. Each EPP system provides an implementation of at least some of the pull functions and/or the push functions. During an EPP, the EPP systems employ the EPP API to exchange data such as electrocardiograms, arrhythmia source locations, catheter routes, electroanatomical mapping data, and electronic health records. The EPP systems may employ aspects of a trusted execution environment and encryption techniques to help ensure integrity of code and data of the EPP systems, authorization of the EPP systems, and security of communications between the EPP systems.

### CROSS REFERENCE TO RELATED APPLICATIONS

This application claims the benefit of U.S. Pro. App. No. 63/375,173 entitled “Enhanced Electronic Medical Record System” and filed on Sep. 9, 2022 and U.S. Pro. App. No. 63/495,475 entitled “Computer Integration Architecture for Cardiology Systems” and filed on Apr. 11, 2023, the disclosures of which are incorporated herein by reference in their entirety.

### BACKGROUND

An electrophysiological (EP) procedure (e.g., an ablation procedure) may be supported by a variety of systems referred to as electrophysiological procedure (EPP) systems. These EPP systems may include a navigation system, a stereotactic ablation radiotherapy (SABR) system, an electroanatomical mapping (EAM) system, an electrocardiogram (ECG) system, an imaging system, an arrhythmia mapping system, a path planning system, an electronic health record (EHR) system, and so on. A navigation system controls movement of a catheter through the heart to a target location based on instructions received typically from an electrophysiologist. Energy is then enabled to the catheter tip to destroy tissue or disrupt cardiac cell function at the target location. The energy may be, for example, thermal energy or electrical energy (e.g., pulsed field). A SABR system delivers radiation from an external radiation source to the target location. A SABR system controls movement of the radiation source to deliver radiation from various positions and angles and at various dosages. An EAM system tracks the location of the catheter tip, creates a three-dimensional (3D) representation of a heart, and records the voltages and timing of electrical activity at locations within the heart. An ECG system collects a continuous recording of an ECG during an EP procedure. An arrhythmia mapping system may input an ECG, identify a portion of the ECG, identify a source location of an arrhythmia based on that portion, and output an indication of the source location. The source location may be the target location for the ablation. An imaging system (e.g., a computed tomography (CT) device) collects images of a patient. A path planning system generates a planned route for movement of a catheter prior to and/or during an EP procedure. An EHR system maintains electronic health records, also known as electronic medical records, of patients.

During an EP procedure, one EPP system may rely on data collected by or generated by another EPP system to perform its function. For example, an arrhythmia mapping system may rely on data of an EHR system, an EAM system, and an ECG system to identify a source location. As another example, a navigation system may rely on data of an arrhythmia mapping system and a path planning system. A path planning system may rely on actual route data generated by a navigation system to dynamically adjust the planned route.

Currently, EPP systems in general cannot communicate directly with each other and employ proprietary formats for their data. For example, an arrhythmia mapping system may not be able to communicate with an ECG system to receive an ECG, with an EAM system to receive an EAM, and with a navigation system to provide a source location and a 3D mesh generated based on the EAM. Because EPP systems may be unable to communicate with each other, data may be transferred via a “sneakernet” by one EPP system storing data onto a storage device (e.g., USB flash drive) connected to it, a person disconnecting the storage device and connecting the storage device to another EPP system, and that EPP system then reading the data from the storage device. The transferring of data using such a manual technique may be prone to error. For example, a USB flash drive may be reused to transfer ECGs during multiple EP procedures. If the USB flash drive stores data of multiple patients, there is a possibility that an EPP system may retrieve the wrong patient's data from the USB flash drive leading to undesirable consequences. Also, since a manual process is involved, the transfer may take longer than if it were automated and is not guaranteed to be done in real time. As a result, the patient may be disadvantaged because the risk may increase with an increased length of an EP procedure and because actions may be taken that are not based on the most current data that is yet to be transferred.

Some attempts have been made to allow the EPP systems to communicate directly. For example, a user interface (UI) system has been developed to allow an EPP system to present and control the UI generated by another EPP system. The UI system may be a dedicated device that receives display data from the other EPP system and then forwards it to the EPP system and receives control data from the EPP system and then forwards it to the other EPP system. In addition, some attempts have been made to define a standard data format for various types of data. For example, OpenEP is open source software that supports analysis of EAM data. (See, openep.io) The software relies on a standard format for the EAM data. Because EAM data is typically stored in proprietary formats by EAM systems, EAM data needs to be converted to the standard format if the software is to be used. The standard format may also be employed to transfer EAM data from one EPP system to another EPP system.

### BRIEF DESCRIPTION OF THE DRAWINGS

FIG. 1 illustrates an example intranet architecture of an EPP integration system.

FIG. 2 is a flow diagram that illustrates example communications between EPP systems.

FIG. 3 is a block diagram that illustrates an architecture of an EPP system that is a cardiology support system.

FIG. 4 illustrates the inputs and outputs of the components of the cardiology support system in some embodiments.

FIG. 5 is a block diagram that illustrates example processing of a navigation system in some embodiments.

### DETAILED DESCRIPTION

An EPP integration system is described that supports the integration of EPP systems. In some embodiments, the EPP integration system provides various EPP communication architectures and provides an EPP application programming interface (API) that support communications between EPP systems. The EPP communication architectures include a direct connection architecture, an intranet architecture, a shared storage architecture, an Internet architecture, and an embedded architecture. In addition, because the storage and use of EHRs need to comply with government regulations such as the Health Insurance Portability and Accountability Act (HIPAA) of the United States and the General Data Protection Regulation (GDPR) of the European Union, the EPP integration system provides data protection for EHRs. The data protection may be based on asymmetric encryption (e.g., using private and public keys of the Rivest-Shamir-Adleman (RSA) standard) or symmetric encryption (e.g., using a symmetric key of the Advanced Encryption Standard (AES)) Also, the EPP integration system helps ensure that code and data of an EPP system can be authenticated and verified as not having been tampered with. For example, any modification to or replacement of code and data (e.g., during a cyberattack) can be detected and prevented from being used. The EPP integration system helps protect the proprietary code and data of the EPP systems.

The EPP API specifies a standard communication protocol and standard data formats that allow EPP systems to communicate (e.g., to exchange data) in real time and securely. The standard communication protocol specifies both pull and push functions for communications between EPP systems. The EPP API specifies the behavior, the input and output parameters, and the formats of the parameters. In general, the EPP API specifies a pull function and a push function for each type of data that can be transferred between EPP systems. In addition, the EPP API specifies a push function and a pull function to transfer a collection of data of different data types or even data of arbitrary data types that are not specified by the EPP API. A pull function is invoked by one EPP system to request data from another EPP system, and a push function is invoked by one EPP system to send data from an EPP system to another EPP system either with or without being requested to do so. The pull and push functions in general have the following names and parameters:

pull “type” (requestor, supplier, patient, procedure, data
type, data format, data characteristics, recurrence)
push “type” (supplier, receiver, patient, procedure, data,
data type, data format, data characteristics, error code)

where “type” in the function name represents the common name of the data type to be sent such as “pullECG” for an electrocardiogram or “pushEAM” for an electroanatomical map. The “requestor,” “supplier,” and “receiver” parameters each represent a unique identifier of an EPP system, and the “patient” and “procedure” parameters represent unique identifiers of a patient and an EPP procedure. The “data” parameter represents the actual data being transferred from one EPP system to another EPP system. The “data type” parameter represents the type of data to be transferred or that is being transferred such as an ECG or an EAM, which may also be implied by the function name. Alternatively, the EPP API may specify only one “pull” and only one “push” function and rely on the “data type” parameter to specify behavior of the functions. The “data format” parameter specifies the format of the data to be transferred such as bitmap image or voltage-time series for an ECG or Visualization Toolkit (VTK) or point cloud for a 3D mesh. The “data characteristics” parameter specifies characteristics of the data to be transferred such as an ECG collected in a certain time range (e.g., specified using the Coordinated Universal Time (UTC)). The data characteristics may be specified using Extensible Markup Language (XML). The “recurrence” parameter specifies conditions that indicate when the supplier should automatically send the requested data to the requestor (e.g., once every minute for ECG data). The data and data characteristics of a push function may each be an array of the data such as an array of ECGs along with their start and end times. The “error code” parameter identifies an error relating to the pulling or pushing of data. For example, the error code may indicate that a patient with the patient identification specified in a pull function cannot be found or that the requested data format is not supported. The parameters may have default and/or null values to indicate, for example, a default format (e.g., Unicode) for the parameter or to indicate the parameter (e.g., procedure identifier) is not being supplied. A message with a specification of a function being called that is sent from a sending EPP system to a receiving EPP system may be encrypted with a public key of the receiving EPP system and signed using the private key of the sending EPP system. Upon receiving the message, the receiving EPP can verify the signature of the sending EPP system using the public key of the sending EPP system and decrypt the message using its private key. An EPP system may publish its public key so that it is generally available to other EPP systems, or an EPP system push its public key to another EPP system, for example, in response to a pull request.

The following API table lists functions defined by the EPP API. The function names are prefixed by “pull” or “push.” However, an implementation of the EPP API may employ different names for functions that provide similar parameters and behaviors.

API TABLE

| Name | Data Type | Data Format | Data Characteristics | Description |
|---|---|---|---|---|
| UIInput | mouse | (x, y) integers | | current position of pointer |
| | key | Unicode | | key most recently pressed |
| | touch | (x, y) integers | | position most recently |
| | | | | touched |
| UIOutput | display | bitmap | resolution | image to be displayed |
| ECG | ECG | array of (v, t) | start time, array size, | patient ECG |
| | | real | voltage units (e.g., mV) | |
| | | bitmap | image of an ECG | |
| | | pdf | pdf vector format | |
| Mesh | 3D mesh | VTK, point | resolution, cardiac portion | 3D mesh representing the |
| | | cloud | (e.g., chamber), color | geometry of the heart |
| | | | scheme(e.g., red for dead | |
| | | | tissue, green for source | |
| | | | location) | |
| Image | Image | DICOM | resolution, coverage (e.g., | an image collected from |
| | | | upper thorax), image type | the patient |
| | | | (e.g., CT) | |
| ABP | source location | array of | | (ablation parameters |
| | area | ((x, y, z), | | “ABP”)area of a source and |
| | | confidence) | | confidence that each |
| | | integers | | coordinate is in the area of |
| | | | | the source |
| | target location | (x, y, z) integers | | target location for the |
| | | | | ablation |
| | target pattern | array of (x, y, z) | | target pattern for the |
| | | integers | | ablation |
| | target lesion | array of | | target area and burn |
| | characteristics | ((x, y, z), burn) | | characteristics |
| | | integers | | |
| | lesion area | array of | | actual area of the lesion |
| | | ((x, y, z,), burn) | | |
| | | integers | | |
| Nav | target route | array of (x, y, z) | | target route of the catheter |
| | | integers | | to from entry to target |
| | | | | location |
| | actual route | array of | | actual route and timing of |
| | | (x, y, z, t) | | catheter movement to its |
| | | integers | | current location |
| EHR | prior ablation | depends on | patient ID, characteristics | characteristics of a prior |
| | | data type | (e.g., date, time, lesion | ablation |
| | | | area) | |
| | tissue state | array of | patient ID | cardiac tissue state |
| | | ((x, y, z), state) | | |
| | | integers | | |
| | . | | | |
| | . | | | |
| | . | | | |
| EAM | EAM | array of | voltage units | array of coordinates and |
| | | ((x, y, z, t), | | voltages of an EAM |
| | | voltage) | | |
| | | integers | | |
| BC | beat | array of | | start and end times of |
| | classification | ((tₛ, tₑ), class) | | beat of an ECG and |
| | | integers | | classification (e.g., |
| | | | | AF or AFL) |
| MD | morphology | array of | | start and end times of beat |
| | delineation | ((tₛ, tₑ), type) | | within an ECG and type of |
| | | integers | | morphology (e.g., QRS) |
| SUB | substrate | array of | | coordinate and substrate |
| | | ((x, y, z), type) | | type (e.g., scar, thickness) |
| | | integers | | |
| PK | public key | integer, type | | encryption public key type |
| | | | | (e.g., AES_256) |

Although not shown, as described above, the EPP API may define a pull function and a push function to allow EPP systems to communicate data of any data type in any format that the EPP systems agree upon. For example, two EPP systems may communicate data in a proprietary format. Pull and push functions may also be employed to send data of multiple data types such as target location, target pattern, and target lesion characteristics in a single function call.

The use of the EPP API allows EPP systems to be replaced by other EPP systems that implement the same functions of the EPP API. A provider of an EPP system may provide two different implementations of that EPP system. For example, one implementation may comply with a certain government regulation but not the other. As another example, one implementation may be based on machine learning but not the other. Also, the EPP system of one provider can be replaced by the EPP system of another provider, for example, because of newly adopted import or export restrictions.

The EPP systems may communicate by sending messages specifying a function and its parameters. The messages may be sent using various communication protocols such as Transmission Control Protocol/Internet Protocol (TCP/IP), Hypertext Transfer Protocol (HTTP), and a Virtual Private Network (VPN). The direct connection architecture supports two EPP systems communicating via a direct wired or wireless connection. The intranet architecture supports a private network (e.g., within a medical facility) to connect (e.g., via an Ethernet network) an arbitrary number of EPP systems. The Internet architecture allows an EPP system to communicate with other EPP systems via the Internet. For example, an ECG system may push ECG data to an EHR system via the Internet. The shared storage architecture allows one EPP system to store a function name and its parameters on shared storage that is accessible by another EPP system that implements the behavior of the function. The other EPP system may periodically monitor the storage to determine the presence of a function name and its parameters and execute the behavior of the function. The embedded architecture system supports computer code and data of one EPP system to be stored on and executed by a computer system executing another EPP system. More generally, any number of EPP systems may execute on the same computing system.

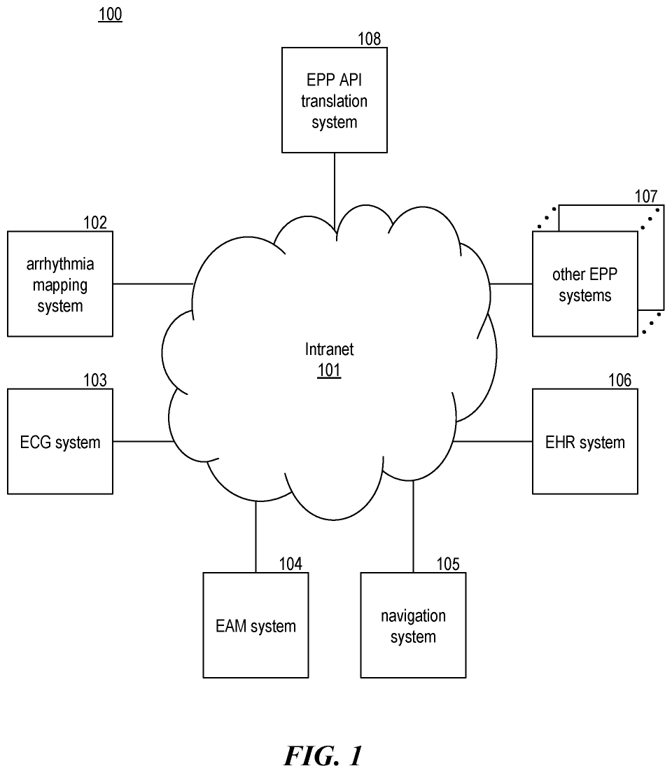

FIG. 1 illustrates an example intranet architecture of the EPP integration system. The intranet architecture 100 includes an intranet 101 such as an Ethernet network. The EPP systems include an arrhythmia mapping (ARM) system 102, an ECG system 103, an EAM system 104, a navigation system 105, an EHR system 106, other EPP systems 107 (e.g., fluoroscopy, ultrasound, and CT systems), and an EPP API translation system 108. The intranet may also be connected to the Internet. The arrhythmia mapping system may receive an arrhythmia ECG (or vectorcardiogram (VCG)) from the ECG system and an EAM from the EAM system and send a source location of the arrhythmia to the navigation system. The arrhythmia mapping system may generate a 3D mesh representing the patient's heart based on the EAM. The 3D mesh may also be generated based on imaging data such as CT scans of the patient's heart. Techniques for generating such a 3D mesh are described in PCT App. No. PCT/US23/14406 entitled “Overall Ablation Workflow System” and filed on Mar. 2, 2023, which is hereby incorporated by reference. The arrhythmia mapping system may run simulations of electrical activity of the patient's heart based on the 3D mesh assuming a variety of source locations of an arrhythmia and generate a simulated ECG (or VCG) for each simulation based on the simulated electrical activity. The arrhythmia mapping system then identifies a source location based on similarity between the received ECG and the simulated ECGs and provides the source location to the navigation system, which generates a planned catheter route for the procedure. Alternatively, a path planning system (not illustrated) may be employed to develop a catheter route. The navigation system may control movement of the catheter based on the planned route. Techniques for identifying a source location are described in U.S. Pat. No. 10,860,754 entitled “Calibration of Simulated Cardiogram” and issued on Dec. 8, 2020, which is hereby incorporated by reference.

The EPP API translation system provides translation of EPP API function invocations to other APIs which may be proprietary. For example, an ECG system may support a proprietary API and not the EPP API. Rather than having the arrhythmia mapping system and the EHR system also implement the proprietary API, a translator may be developed to translate between the EPP API and the proprietary API. The EPP API translation system thus functions as an intermediary between EPP systems that support the EPP API and those that do not.

In some embodiments, the Internet architecture supports some or all of the EPP systems communicating via the Internet. For example, the intranet of FIG. 1 may have a connection to the Internet so that the EPP systems can communicate with an EPP system not connected to the intranet. For example, an EPP system may be a billing system that is only accessible via the Internet. Before, during, or after a procedure, an EPP system connected to the intranet may send billing information to the billing system via the Internet. As another example, an EPP system may pull imaging data from an imaging system that is only accessible via the Internet. The Internet communication architecture may also support all EPP system communicating via the Internet without communicating via an intranet. (Note: The term “EPP system” refers to any system that may be accessed in conjunction with an EP procedure—before, during, or after.)

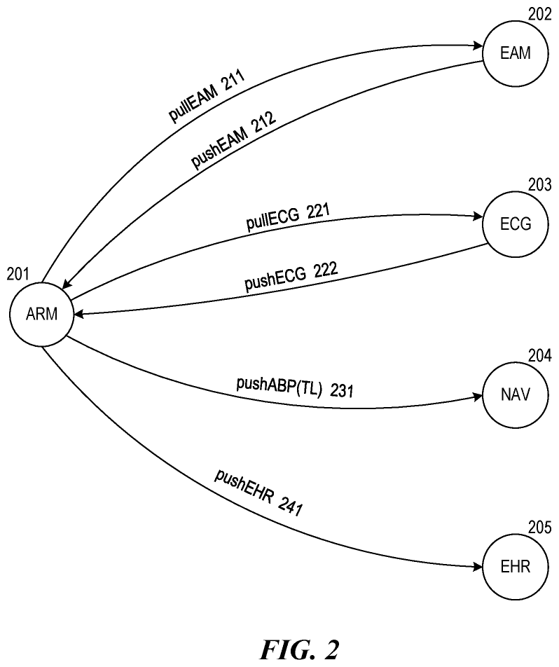

FIG. 2 is a flow diagram that illustrates example communications between EPP systems. In this example, an ARM system 201 communicates with an EAM system 202, an ECG system 203, a navigation (NAV) system 204, and an EHR system 205. The ARM system initially sends 211 to the EAM system a pullEAM request for the patient's EAM. In response, the EAM system retrieves an EAM and sends 212 to the ARM system a pushEAM response with the EAM. The ARM system may then generate a 3D mesh of the patient's heart based on the EAM. The ARM system may also run simulations of electrical activity of the patient's heart based on the 3D mesh assuming a variety of simulated source locations and generate a simulated ECG for each simulation that is mapped to a simulated source location as described in U.S. Pat. No. 10,319,144 entitled “Computational Localization of Fibrillation Sources” and issued on Jun. 11, 2019, and U.S. Pat. No. 10,860,754, which are hereby incorporated by reference. The ARM system then sends 221 to the ECG system a pullECG request to retrieve an ECG of the patient. In response, the ECG system retrieves an ECG and sends 222 to the ARM system a pushECG response with the ECG. The ARM system may identify a portion of the ECG based on manual or automatic selection for use in identifying a source location and/or generating a classification of an ECG such as normal or abnormal, left or right chamber, or endocardial or epicardial. The ARM system then identifies a simulated source location to be used as a target source location based on similarity between the patient's ECG and the simulated ECGs, for example, using a machine learning model as described in U.S. Pat. No. 10,860,754. The ARM system then sends 231 to the navigation system a pushTL request with the target source location. In response, the navigation system may control a catheter to move the catheter tip to the target source location. The ARM system sends 241 to the EHR system a pushEHR request to store, for example, the target source location as part of the patient's EHR. In response, the EHR system stores the target source location as part of the patient's EHR.

An embedded architecture system supports computer code and data (referred to as trusted code and trusted data) of an embedded EPP system to be stored on and executed by a host EPP system. To help ensure that the trusted code and trusted data of the embedded EPP system have not been tampered with, the embedded EPP system may be executed in a Trusted Execution Environment (TEE) of the processor of the host EPP system. For example, the code and data of an arrhythmia mapping system may be stored in protected memory of and executed in a TEE of a navigation system. A TEE is a feature of a central processing unit (“CPU”) in which trusted code and trusted data are stored in an enclave. An enclave is protected memory in which trusted code and trusted data are stored in encrypted form and decrypted by the TEE only when retrieved for execution or use. Untrusted code, which is code other than the trusted code, cannot inspect or interfere with the execution of the trusted code (at least not without the permission of the trusted code). The TEE thus protects trusted code and trusted data while at rest (within the enclave), in motion (between the TEE and the enclave), and during execution (within the TEE). The Intel Software Guard Extensions (SGX) and the ARM TrustZone are examples of a TEE. In the following, a TEE is described primarily in the context of SGX.

During manufacture of a processor, the TEE is provided with a set of CPU private keys. The CPU private keys are stored in such a way that they cannot be altered (e.g., deleted) in any way. The corresponding CPU public keys are stored by the manufacturer. The CPU supports generating an attestation of the trusted code of the enclave. The attestation includes a hash of the trusted code, an identifier of the CPU, and the trusted data. The attestation is signed by a CPU private key. A host EPP system can request the CPU to provide the attestation as evidence of the trusted code and trusted data stored in the enclave. (The terms “client” and “server” in this context refer to the embedded EPP system and an EPP system that requests services of the embedded EPP system.) The client can request a service provided by the manufacturer of the CPU to verify the signature using the corresponding public key. The client can then verify that the hash is a hash of the trusted code and the trusted data that the client expects.

To persistently store data on behalf of client code that accesses services of the trusted code, the TEE creates an encryption key and a decryption key that may be a public/private keypair (or a symmetric key) and provides the encryption key to the client code. (The attestation may also include a public key that the client can use to encrypt data that the TEE can decrypt using the corresponding private key.) When the TEE stores data on behalf of the client, the TEE encrypts the data with the encryption key and stores the data in memory accessible to the client. The client code can retrieve and decrypt that encrypted data with the decryption key. Similarly, the client can also encrypt data that it sends to the TEE or stores for access by the TEE using the decryption key. Because only the TEE has the encryption key (or the TEE and the client in the case of a symmetric key), only the TEE can decrypt the encrypted data. Alternatively, the TEE may generate a public/private keypair for data to be encrypted by the TEE and a private/public keypair for data to be encrypted by the client. Further details of a TEE are provided below.

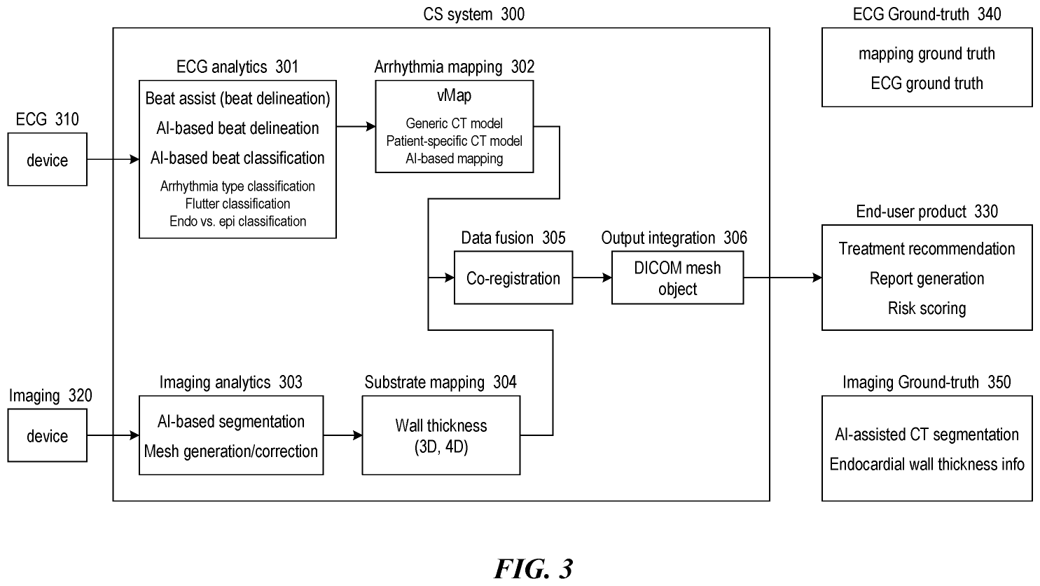

FIG. 3 is a block diagram that illustrates an architecture of an EPP system that is a cardiology support system. The cardiology support (CS) system 300 includes an ECG analytics component 301, an arrhythmia mapping component 302, an imaging analytics component 303, a substrate mapping component 304, a data fusion component 305, and an output integration component 306. The CS system interfaces with other EPP systems such as an ECG system 310, an imaging system 320, an end-user product system 330, an ECG ground-truth system 340, and an imaging ground-truth system 350. Each component receives input from another EPP system or another component, processes the input to generate an output, and sends the output to another EPP system or another component.

The ECG analytics component 301 provides processing functions for inputting an ECG, analyzing an ECG, and outputting the results of the analysis. The processing functions may include beat delineation (AI-based or not AI-based) within an ECG and AI-based beat classification of beats (cycles) of the ECG. The ECG analytics system inputs an ECG from various systems such as ECG system 310. A beat delineation function supports the displaying of an ECG and the providing of assistance to a user in selecting a beat of the ECG that is to be used in evaluating various conditions such as whether the beat represents normal or abnormal electrical activity of a heart (e.g., normal sinus rhythm (NSR) or AF). An AI-based beat delineation function employs a beat delineation machine learning (ML) model to delineate a beat, which may be manually adjusted. An AI-based beat classification function employs a beat classification ML model that inputs an ECG (or an individual beat) and outputs various characteristics of the ECG such as arrhythmia type (e.g., AF or atrial flutter (AFL)), flutter type (e.g., atypical), or an endocardial or epicardial classification. The AI-based beat classification function may also base the beat classification on patient characteristics (e.g., prior ablation location) that are provided by an EHR system. Techniques for delineating a beat, identifying arrhythmia type, and generating an endocardial or epicardial classification are described in PCT App. No. PCT/US23/73866 entitled “Automatic Refinement of Electrogram Selection” and filed on Aug. 24, 2023; PCT App. No. PCT/US23/72854 entitled “Automatic Fibrillation Classification and Identification of Fibrillation Epochs and filed on Aug. 23, 2022, U.S. Pro. App. No. 63/446,324 entitled “Atrial Flutter Classification System” and filed on Feb. 16, 2023, and U.S. Pro. App. No. 63/516,911 entitled “Heart Wall Refinement of Arrhythmia Source Locations” and filed on Aug. 1, 2023, which are hereby incorporated by reference. ECG systems that provide input to the ECG analytics component may include cardiology information systems, ablation procedure support systems, clinical ECG devices, EHR systems, portable ECG monitoring devices (e.g., Holter monitor), ECG-enabled smartwatches, and so on. Some ECG systems are described in U.S. Pro. App. No. 63/412,830 entitled “Body Composition and ECG Processing System” and filed on Oct. 3, 2022, which is hereby incorporated by reference. The ECG analytics component may provide a conversion function to convert an ECG image to a voltage-time series representation. Techniques for such conversion are described in PCT App. No. PCT/US23/22146 entitled “Encoding Electrocardiogramata” and filed on May 12, 2023, which is hereby incorporated by reference.

An arrhythmia mapping component 302 provides processing functions to support identifying the source location of an arrhythmia based on one or more beats (or portions of beats) of an ECG. The arrhythmia mapping system may also identify other information that may be helpful in treating a patient such as an ablation pattern, an ablation target location, a parameter plan for an ablation procedure, and so on. A source location identification function may be AI-based or not AI-based. When not AI-based, the source location identification function may access mappings of beats to source locations to identify a source location based on similarity of a beat to the patient's beat. When AI-based, the source location identification function may access a mapping ML model that is trained using beats that are labeled with source locations (derived from the mappings) to identify a source location given a patient's beat. Techniques for generating a parameter plan are in described U.S. Pro. App. No. 63/496,366 entitled “Ablation Targeting and Planning System” and filed on Apr. 14, 2023, which is hereby incorporated by reference.

The mappings may be generated based on running simulations of cardiac electrical activity assuming simulated or clinical characteristics of a heart such as cardiac geometry, electrical characteristics (e.g., conduction velocity), source locations, and so on and deriving an ECG from the simulated cardiac electrical activity. Such simulations are considered to be based on generic cardiac models because they are not based on characteristics derived from the patient. A generic cardiac model may be based on simulated characteristics such as cardiac geometry and electrical characteristics or may be based on clinical characteristics collected from EHRs of other patients such as cardiac geometries derived from 3D images (e.g., CT images) of those other patients. The mappings may also be generated without running simulations such as by deriving the mappings from EHRs of many patients.

The mappings may also be patient specific in that they are generated based on patient-specific simulations that are based on patient-specific characteristics of the patient such as a cardiac geometry derived from a 3D image of the patient or a known scar location of the patient. For example, the arrhythmia mapping system may run patient-specific simulations of cardiac activity based on the cardiac geometry of the patient and/or train a source location ML model using data identified based on patient characteristics of the patient. A patient-specific simulation may be based on both patient-specific characteristics and non-patient specific characteristics. For example, the simulation may be based on a simulated cardiac geometry and a patient-specific scar tissue. Techniques for arrhythmia mapping are described in U.S. Pat. Nos. 10,319,144 and 10,860,754. Techniques for identifying other information are described in U.S. Pat. No. 11,383,131 entitled “Guiding Implantation of an Energy Component in a Body” and issued on May 24, 2022, and U.S. Pat. No. 11,534,224 entitled “Interactive Ablation Workflow System” and issued on Dec. 27, 2022, which are hereby incorporated by reference.

The imaging analytics component 303 provides processing functions that may provide image segmentation and mesh generation. An image segmentation function inputs a cardiac 3D image (e.g., CT image or magnetic resonance imaging (MRI) image) from an imaging system (or EHR system) and generates a segmentation of the cardiac image. The segmented 3D image may identify cardiac structures such as the left ventricle or right atrium. For example, each slice of a CT image may have the portion of a slice corresponding to the right atrium demarcated and indicated as corresponding to the right atrium. A mesh generation function may input a segmented CT image of a heart and generate a 3D mesh representing the heart. A 3D mesh includes vertices, edges, and faces based on a cardiac geometry derived from the 3D image. The vertices (e.g., 70,000) represent locations throughout the cardiac wall from the endocardium through the epicardium. Techniques for segmenting a 3D image and generating a 3D mesh are described in PCT App. No. PCT/US23/14406. The imaging analytics component may also provide a user interface through which a user can review a 3D graphic based on the 3D mesh along with its segmentation and revise both the geometry of the 3D mesh and the segmentation.

The substrate mapping component 304 provides processing functions to determine cardiac wall thickness of a heart and scar tissue within the heart given a 3D mesh representing the heart. A wall thickness function determines the thickness of a cardiac wall based on the distance between the endocardium and pericardium or epicardium. The wall thickness function identifies vertices of the 3D mesh corresponding to the locations within the endocardium and epicardium and determines wall thickness based on distances between vertices of the endocardium and vertices of the epicardium. The wall thickness function may also identify scar tissue based on an analysis of the wall thickness. Techniques for determining wall thickness are described in U.S. Pro. App. No. 63/492,772 entitled “Heart Wall Thickness System” and filed on Mar. 28, 2023, which is hereby incorporated by reference. The image analytics and substrate mapping components may also provide other functions such as identifying tissue state (e.g., levels of perfusion) and adding tissue state information to a 3D mesh. Techniques for providing such other functions are described in U.S. application Ser. No. 17/882,426 entitled “Tissue State Graphic Display System” and filed on Aug. 5, 2022, which is hereby incorporated by reference.

The data fusion component 305 provides processing functions to fuse or co-register the data generated by the arrhythmia mapping component with data generated by the substrate mapping component. For example, when the arrhythmia mapping component is based on a generic 3D mesh (i.e., not patient specific), the source location identified by the arrhythmia mapping component is relative to the generic 3D mesh such as at a certain vertex. However, because of differences in cardiac geometries between the generic 3D mesh and the patient-specific 3D mesh (e.g., the patient has an enlarged right ventricle), the source location of the arrhythmia may be more accurately represented as being at a different vertex of the patient-specific 3D mesh. A co-registration function adjusts the source location to be relative to the patient-specific 3D mesh and adds an indication of the source location to the patient-specific 3D mesh. Techniques for adjusting a source location based on differences between a generic 3D mesh and a patient-specific 3D mesh are described in PCT App. No. PCT/US23/14406.

The output integration component 306 provides a processing function to convert the fused data to formats of the EPP API to provide to other EPP systems. A DICOM mesh object function converts a 3D mesh to a Digital Imaging and Communications in Medicine (DICOM) formatted object. Techniques for such converting are described in U.S. Pat. No. 10,952,794 entitled “Augmentation of Images with Source Locations” and issued on Mar. 23, 2021, which is hereby incorporated by reference.

The CS system may output a 3D mesh to various end-user product systems 330 for further processing. One type of end-user product system is an arrhythmia assessment system that provides, for example, an assessment of life expectancy with and without an ablation procedure, the chance of having a stroke, the success rate of ablation, and so on. Techniques for providing such an assessment are described in U.S. Pro. App. No. 63/450,593 entitled “Arrhythmia Assessment Machine Learning” and filed on Mar. 7, 2023, which is hereby incorporated by reference.

An ECG ground-truth system 340 allows a medical provider to indicate a successful beat, ablation location, and ablation pattern that was used in a successful procedure. The successful beat may have been a beat identified by the ECG analytics component or a beat manually delineated by a medical provider and provided directly to the arrhythmia mapping component. Successful beats can then be used to improve the ECG analytics component, for example, by retraining a beat delineation ML model using successful beats as training data. Successful target locations and ablation patterns can be used to improve the arrhythmia mapping component, for example, by retraining a mapping ML model using the successful target locations and ablation patterns as training data.

An imaging ground-truth system 350 allows a medical provider to generate a segmented 3D image manually or using a segmentation ML model to assist in segmentation or to revise a segmented 3D image generated by the imaging analytics component. These segmented 3D images may be used to improve the segmentation of the imaging analytics component, for example, by retraining a segmentation ML model using the segmented 3D images as training data. Another imaging ground-truth system allows a medical provider to generate wall thickness information or to correct wall thickness information provided by the substrate mapping component. This wall thickness information may be used to improve the substrate mapping component. For example, the substrate mapping component may employ a wall thickness ML model to identify wall thicknesses given a 3D mesh which can be retrained based on the wall thickness information. Data generated by the ECG ground-truth system and the imaging ground-truth system may be used as training data to improve a ML model used by the data fusion component.

The various components and functions of the CS system may be employed as standalone EPP systems that integrate with various EPP systems or other external systems. For example, the arrhythmia mapping component may input a beat directly from an ECG system, identify a source location, and output the source location to a navigation system. As another example, the imaging analytics component may input a 3D image from an imaging system, generate a segmented 3D image, and output the segmented 3D image to a navigation system. Rather than individual components being standalone EPP systems, multiple components may be combined to form a combined EPP system. For example, the imaging analytics component, the substrate mapping component, and the output integration component may form a pipeline that inputs a 3D image from an imaging system, generates a 3D mesh, annotates or demarcates the 3D mesh with wall thickness, converts the 3D mesh to a DICOM format, and outputs the 3D mesh in the DICOM format to a navigation system.

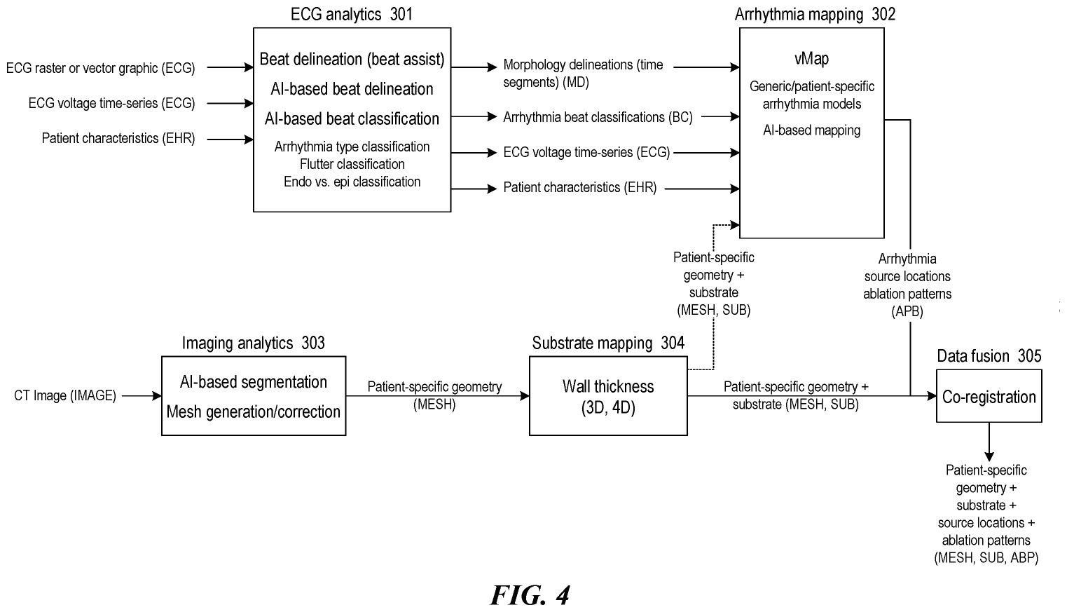

FIG. 4 illustrates the inputs and outputs of the components of the CS system in some embodiments. Each component has pull and push functions for inputting data into and outputting data from a component. The functions and their parameters may be considered part of the EPP API as described above in the API table. The labels associated with arrows indicate the type of data and the function name for pulling or pushing the data.

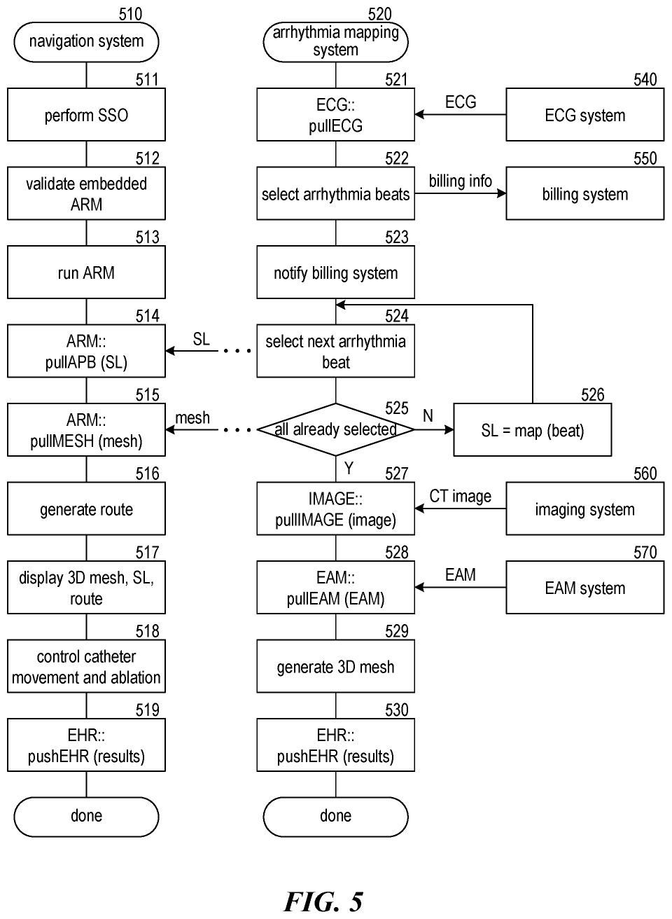

FIG. 5 is a block diagram that illustrates example processing of a navigation system in some embodiments. The navigation system 510 interfaces with arrhythmia mapping system 520, which interfaces with ECG system 540, billing system 550, imaging system 560, and EAM system 570. The navigation system provides the function of generating a planned catheter route and guiding a catheter based on the planned catheter route. The arrhythmia mapping system is embedded code and data stored in an enclave and executed in a TEE of a computer on which the navigation system executes. In block 511, the navigation system performs a single sign-on (SSO). A medical provider signs onto the navigation system. The navigation system then interacts with an SSO server to verify the sign-on credentials and to record the sign-on. When the arrhythmia mapping system is to be run, rather than requesting the medical provider to enter sign-on credentials for the arrhythmia mapping system, the arrhythmia mapping system coordinates with the SSO server to verify that the SSO server has the sign-on credentials for the arrhythmia mapping system. In block 512, the navigation system validates the code and data of the arrhythmia mapping system using the attestation feature of the TEE. In block 513, the navigation system requests the arrhythmia mapping system to generate a mapping for a specified patient. After the arrhythmia mapping is complete, in block 514, the navigation system pulls the source location from the arrhythmia mapping system. In block 515, the navigation system pulls a 3D mesh of the patient from the arrhythmia mapping system. In block 516, the navigation system generates a planned catheter route based on the 3D mesh and the source location. In block 517, the navigation system displays a graphic of the patient's heart based on the 3D mesh that includes an indication of the catheter route and the source location. In block 518, the navigation system controls movement of the catheter to the source location so that the ablation can be performed. In block 519, the navigation pushes results of the ablation procedure to an EHR system and then completes.

In block 521, the arrhythmia mapping system pulls an ECG of the patient from the ECG system. In block 522, the arrhythmia mapping system selects arrhythmia beats of the ECG. The arrhythmia mapping system may allow a medical provider to select the beats. The arrhythmia mapping system may also display an intra-cardiogram similarity graphic user interface to assist a medical provider in evaluating the ECG, for example, as described in U.S. Pat. No. 10,595,736 entitled “Heart Graphic Display System” and issued on Mar. 24, 2020, which is hereby incorporated by reference. In block 523, the arrhythmia mapping system sends billing information relating to use of the arrhythmia mapping system to the billing system. In block 524, the arrhythmia mapping system selects the next beat. In decision block 525, if all the beats have already been selected, then the arrhythmia mapping system continues at block 527, else the arrhythmia mapping system continues at block 526. In block 526, the arrhythmia mapping system maps the beat to a source location. The arrhythmia mapping system may perform an auto-refinement of the beat before mapping. The arrhythmia mapping system may also map the beat to an ablation pattern as described in U.S. Pat. No. 10,860,754. The arrhythmia mapping system may also retrieve a text description for the source location such as an identification of a segment of the AHA model of the American Heart Association such as the “mid anteroseptal” segment. The arrhythmia mapping system then loops to block 524 to select the next beat. In block 527, the arrhythmia mapping system pulls a CT image from the imaging system. In block 528, the arrhythmia mapping system pulls an EAM from the EAM system. In block 529, the arrhythmia mapping system generates a 3D mesh based on the CT image and/or the EAM. In block 530, the arrhythmia mapping system pushes the results of the mapping to an EHR system and then completes. Although not illustrated, the arrhythmia mapping system includes an implementation of a pullABP function and pullMESH function that are invoked by the navigation system.

Additional details of a TEE are provided in the following paragraphs. An enclave includes trusted code and trusted data and a certificate of the author of the enclave. The certificate is referred to as an enclave signature (SIGSTRUCT). The enclave signature includes an enclave measurement, the author's public key, a security version number (ISVSVN) of the enclave, and a product ID (ISVPRODID) of the enclave. The enclave signature is signed using the author's private key. The enclave measurement is a hash of the initial trusted code and the initial trusted data. When trusted code and trusted data are loaded into the enclave (protected memory (EPC)), the CPU calculates a measurement and stores it in an MRENCLAVE register. If the calculated measurement is not equal to the enclave measurement, the CPU will not allow the enclave to be initialized within the TEE. After the enclave is initialized, the CPU stores a hash of the author's public key in an MRSIGNER register as an identifier of the author. The ISVSVN specifies the security level of the enclave. The ISVPRODID identifies the product the enclave represents. The CPU records both the ISVSVN and the ISVPRODID.

An EPP system client that is to interact with an enclave (that has been initialized) would typically require the TEE to “attest” to the trusted code and trusted data of the enclave. To provide an attestation to an EPP system that may be executing on a platform (e.g., computer system) that is different from the platform of the CPU that is executing the enclave (referred to as “remote” attestation), the TEE generates a “report” that includes the measurement (MRENCLAVE), hash of the author's public key (MRSIGNER), attributes of the enclave, and user data of the enclave. The report is passed to a quoting enclave (QE) to verify and sign the report. When verified, the QE generates a “quote” that includes the report and a signature of the TEE. The quote is then sent to the client.

Upon receiving a quote, the EPP system can verify the signature and if verified, ensure that the report represents the trusted code that the EPP system expects. The signature may be based on an enhanced privacy ID (EPID), in which different TEE have different private keys, but signatures based on those private keys can be verified using the same public key. The EPP system may invoke the services of an EPID verification service to verify a signature on a quote.

The trusted code of an enclave that is to interact with trusted code of another enclave that is executing on the same platform may want the other trusted code to attest to its trusted code and trusted data. In such a case, a simplified version of attestation can be used (referred to as “local” attestation). To initiate an attestation, the requesting enclave can send its MRENCLAVE measurement to an attesting enclave. The attesting enclave requests the CPU to generate a report destined for the requesting enclave identified by the MRENCLAVE measurement that it received, and the attesting enclave sends the report to the requesting enclave. The requesting enclave then asks the CPU to verify the report. The attesting enclave may request the requesting enclave to provide an attestation to effect a mutual attestation.

A TEE provides support for an enclave to encrypt data that is to be stored outside of the TEE and to decrypt the encrypted data when it is later retrieved into the TEE. This encrypting and decrypting is referred to as “sealing” and “unsealing.” The TEE generates an encryption key and a decryption key based on a “fused key” that is not known outside of the hardware. The fused key is fused into the CPU hardware during the manufacturing process of the CPU, is not known outside of the CPU not even by the manufacturer, is unique to the CPU, and cannot be accessed except by the hardware. Upon request, the CPU generates a sealing key and an unsealing key (e.g., public/private keypair) that are based on the fused key and data associated with the requesting enclave. Thus, each sealing key and each unsealing key is unique to the CPU because the fuse keys are unique.

The CPU can generate two types of keys based on the associated data of the enclave that is used when generating the keys. The associated data is the MRENCLAVE (referred to as “sealing to the enclave”) or the combination of the MRSIGNER, ISVSVN, and ISVPRODID (referred to as “sealing to the author”). Data that is sealed to the enclave can only be unsealed by an enclave with the same MRENCLAVE value that is executing on the same CPU (i.e., using the same fused key) that generated the sealing key. Data that is sealed to the author can be unsealed by any enclave (e.g., different trusted code) of the author that has the same ISVPRODID and the same or an earlier ISVSVN (specified in a request to seal or unseal) and that is executing on the same CPU (i.e., using the same fused key) that generated the sealing key. (Note: The CPU will not generate seal-to-the-author keys for an ISVSVN that is greater than the ISVSVN of the enclave, which allows for only backward compatibility of sealing.) The TEE provides a seal application programming interface (API) for sealing data and an unseal API for unsealing data.

An EPP system may help ensure that its trusted code and trusted data can only be fully executed by an authorized CPU. The trusted data may include the CPU public key of the TEE of an authorized CPU. Such CPU public keys can be retrieved by the owner of the authorized CPU from the services of the CPU manufacturer. When the trusted code starts execution within a TEE, the trusted code can request the TEE to encrypt original target data provided by the trusted code using the CPU private key. The trusted code then decrypts the encrypted target data using the CPU public key. If the decrypted target data matches the original target data, the CPU is verified as an authorized CPU and the trusted code continues its execution. Otherwise, the trusted code aborts its execution.

The computing systems (e.g., network nodes or collections of network nodes) on which the EPP systems and the other described systems may be implemented may include a central processing unit, input devices, output devices (e.g., display devices and speakers), storage devices (e.g., memory and disk drives), network interfaces, graphics processing units, communications links (e.g., Ethernet, Wi-Fi, cellular, and Bluetooth), global positioning system devices, and so on. The input devices may include keyboards, pointing devices, touch screens, gesture recognition devices (e.g., for air gestures), head and eye tracking devices, microphones for voice recognition, and so on. The computing systems may include high-performance computing systems, distributed systems, cloud-based computing systems, client computing systems that interact with cloud-based computing system, desktop computers, laptops, tablets, e-readers, personal digital assistants, smartphones, gaming devices, servers, and so on. The computing systems may access computer-readable mediums that include computer-readable storage mediums and data transmission mediums. The computer-readable storage mediums are tangible storage means that do not include a transitory, propagating signal. Examples of computer-readable storage mediums include memory such as primary memory, cache memory, and secondary memory (e.g., DVD), and other storage. The computer-readable storage mediums may have recorded on them or may be encoded with computer-executable instructions or logic that implements the EPP system and the other described systems. The data transmission mediums are used for transmitting data via transitory, propagating signals or carrier waves (e.g., electromagnetism) via a wired or wireless connection. The computing systems may include a secure crypto processor as part of a central processing unit (e.g., Intel Software Guard Extensions (SGX)) for generating and securely storing keys, for encrypting and decrypting data using the keys, and for securely executing all or some of the computer-executable instructions of the EPP systems.

The one or more computing systems may include client-side computing systems and cloud-based computing systems (e.g., public or private) that each execute computer-executable instructions of the EPP systems. A client-side computing system may send data to and receive data from one or more servers of the cloud-based computing systems of one or more cloud data centers. For example, a client-side computing system may send a request to a cloud-based computing system to perform tasks such as run a patient-specific simulation of electrical activity of a heart or train a patient-specific ML model. A cloud-based computing system may respond to the request by sending to the client-side computing system data derived from performing the task such as a source location of an arrhythmia. The servers may perform computationally expensive tasks in advance of processing by a client-side computing system such as training an ML model or in response to data received from a client-side computing system. A client-side computing system may provide a user experience (e.g., user interface) to a user of the EPP systems. The user experience may originate from a client computing device or a server computing device. For example, a client computing device may generate a patient-specific graphic of a heart and display the graphic. Alternatively, a cloud-based computing system may generate the graphic (e.g., in a Hypertext Markup Language (HTML) format or an Extensible Markup Language (XML) format) and provide it to the client-side computing system for display. A client-side computing system may also send data to and receive data from various medical devices such as an ECG system, an ablation therapy device, an ablation planning device, and so on. The data received from the medical devices may include an ECG, actual ablation characteristics (e.g., ablation location and ablation pattern), and so on. The data sent to a medical device may include data, for example, data in a Digital Imaging and Communications in Medicine (DICOM) format. A client-side computing device may also send data to and receive data from medical computing systems that store patient medical history data, descriptions of medical devices (e.g., type, manufacturer, and model number) of a medical facility, results of procedures, and so on. The term “cloud-based computing system” may encompass computing systems of a public cloud data center provided by a cloud provider (e.g., Azure provided by Microsoft Corporation) or computing systems of a private server farm (e.g., operated by the provider of an EPP system).

The EPP systems and the other described systems may be described in the general context of computer-executable instructions, such as program modules and components, executed by one or more computers, processors, or other devices. Generally, program modules or components include routines, programs, objects, data structures, and so on that perform tasks or methods or that implement data types of the EPP systems and the other described systems. Typically, the functionality of the program modules may be combined or distributed as desired in various examples. Aspects of the EPP systems and the other described systems may be implemented in hardware using, for example, an application-specific integrated circuit (ASIC), a graphics processing unit (GPU), or a field programmable gate array (FPGA).

The following paragraphs describe various embodiments of aspects of the EPP integration system and other systems. An implementation of the systems may employ any combination of the embodiments. The processing described below may be performed by a computing system with a processor that executes computer-executable instructions stored on a computer-readable storage medium that implements the system.

In some aspects, the techniques described herein relate to a plurality of electrophysiological procedure systems that support an electrophysiological procedure and that implement an electrophysiological procedure application programming interface, the plurality of electrophysiological procedure systems including: a first electrophysiological procedure system and a second electrophysiological procedure system wherein the first electrophysiological procedure system provides first implementations of one or more pull functions having parameters specifying data to be provided by the first electrophysiological procedure system to an invoking electrophysiological procedure system and one or more push functions having parameters specifying data being provided by an invoking electrophysiological procedure system to the first electrophysiological procedure system; wherein the second electrophysiological procedure system provides second implementations of one or more pull functions having parameters specifying data to be provided by the second electrophysiological procedure system to an invoking electrophysiological procedure system and one or more push functions having parameters specifying data being provided by an invoking electrophysiological procedure system to the second electrophysiological procedure system; wherein the electrophysiological procedure application programming interface specifies behavior and parameters of push functions and behavior and parameters of pull functions so that electrophysiological procedure systems that implement push functions and pull functions can exchange data during an electrophysiological procedure; wherein the first electrophysiological procedure system includes computer-executable instructions for controlling one or more computing systems to execute the first implementations; and wherein the second electrophysiological procedure system includes computer-executable instructions for controlling one or more computing systems to execute the second implementations. In some aspects, the techniques described herein relate to a plurality of electrophysiological procedure systems wherein the first electrophysiological procedure system can exchange data with a third electrophysiological procedure system rather than the second electrophysiological procedure system when the third electrophysiological procedure system provides third implementations of the same pull functions and pull functions provided by the second electrophysiological procedure system. In some aspects, the techniques described herein relate to a plurality of electrophysiological procedure systems wherein the first electrophysiological procedure system and the second electrophysiological procedure system communicate via one or more of a direct connection architecture, an intranet architecture, a shared storage architecture, an Internet architecture, and an embedded code architecture. In some aspects, the techniques described herein relate to a plurality of electrophysiological procedure systems wherein the computer-executable instructions of the first electrophysiological procedure system are executed by a first computing system and the computer-executable instructions of the second electrophysiological procedure system execute in a trusted execution environment of the first computing system. In some aspects, the techniques described herein relate to a plurality of electrophysiological procedure systems wherein a trusted execution environment of a computer system provides to the first electrophysiological procedure system an attestation of the computer-executable instructions of the second electrophysiological procedure system. In some aspects, the techniques described herein relate to a plurality of electrophysiological procedure systems wherein data exchanged between the first electrophysiological procedure system and the second electrophysiological procedure system is encrypted data of a push function that is encrypted using public/private key encryption.

In some aspects, the techniques described herein relate to a plurality of electrophysiological procedure systems wherein the electrophysiological procedure application programming interface specifies a pull function and a push function for user interface input data, user interface output data, electrocardiogram data, mesh data, image data, ablation parameter data, navigation data, and electroanatomical mapping data. In some aspects, the techniques described herein relate to a plurality of electrophysiological procedure systems wherein the electrophysiological procedure application programming interface specifies parameters for a pull function and a push function that include data type, data format, and data characteristics. In some aspects, the techniques described herein relate to a plurality of electrophysiological procedure systems wherein the electrophysiological procedure application programming interface specifies parameters for a pull function that include a requestor and parameters for a push function that include a supplier. In some aspects, the techniques described herein relate to a plurality of electrophysiological procedure systems wherein the first electrophysiological procedure system verifies that it is executing on a computing system that it is authorized to execute on based on decrypting data encrypted by a trusted execution environment of the computing system, the data encrypted with a private key of a public/private keypair of the trusted execution environment and decrypted with a public key of the public/private keypair. In some aspects, the techniques described herein relate to a plurality of electrophysiological procedure systems wherein the electrophysiological procedure application programming interface specifies encryption of data to comply with the Health Insurance Portability and Accountability Act and/or the General Data Protection Regulation.

In some aspects, the techniques described herein relate to a method performed by one or more computing systems for executing computer-executable code of a first electrophysiological procedure system and a second electrophysiological procedure system, the method including: storing first computer-executable code of the first electrophysiological procedure system in memory of the one or more computing systems, the first computer-executable code implementing one or more functions of an electrophysiological procedure application programming interface; storing second computer-executable code of the second electrophysiological procedure system in protected memory of a trusted execution environment of the one or more computing systems, the second computer-executable code implementing one or more functions of the electrophysiological procedure application programming interface; and under control of the first computer-executable code, requesting from the trusted execution environment an attestation of the second computer-executable code; receiving from the trusted execution environment the attestation; verifying based on the attestation that the second computer-executable code is as expected; and invoking an invoked function of the electrophysiological procedure application programming interface implemented by the second computer-executable code; wherein the computer-executable code of the invoked function is executed within the trusted execution environment and wherein the electrophysiological procedure application programming interface specifies behavior and parameters of push functions and behavior and parameters of pull functions so that electrophysiological procedure systems that implement push functions and pull functions can exchange data during an electrophysiological procedure. In some aspects, the techniques described herein relate to a method wherein the computer-executable code of the first electrophysiological procedure system and the computer-executable code of the second electrophysiological procedure system execute on different computing systems.

In some aspects, the techniques described herein relate to a method performed by one or more computing systems during an ablation procedure for a patient, the method including: under control of an arrhythmia mapping system that implements an electrophysiological procedure application programming interface, sending to an electrocardiogram system a first request to invoke an electrocardiogram pull function of the electrophysiological procedure application programming interface to retrieve an electrocardiogram of the patient; receiving from the electrocardiogram system a second request to invoke an electrocardiogram push function of the electrophysiological procedure application programming interface, the second request specifying an electrocardiogram of the patient; identifying a source location of an arrhythmia based on the electrocardiogram of the patient; and sending to a path planning system a third request to invoke an ablation parameter push function of the electrophysiological procedure application programming interface, the third request specifying the source location; under control of the electrocardiogram system, receiving from the arrhythmia mapping system the first request to invoke the electrocardiogram pull function; retrieving an electrocardiogram of the patient; sending to the arrhythmia mapping system the second request to invoke the electrocardiogram push function; and under control of the path planning system, receiving from the arrhythmia mapping system the third request to invoke the ablation parameter push function; and generating catheter route for the ablation procedure based on the source location wherein the electrophysiological procedure application programming interface specifies behavior and parameters of push functions and behavior and parameters of pull functions so that electrophysiological procedure systems that implement push functions and pull functions can exchange data during an electrophysiological procedure. In some aspects, the techniques described herein relate to a method wherein the electrophysiological procedure application programming interface specifies encryption of data to comply with the Health Insurance Portability and Accountability Act and/or the General Data Protection Regulation. In some aspects, the techniques described herein relate to a method wherein the arrhythmia mapping system verifies that it is executing on a computing system that it is authorized to execute on based on decrypting data encrypted by a trusted execution environment of the computing system, the data encrypted with a private key of a public/private keypair of the trusted execution environment and decrypted with a public key of the public/private keypair. In some aspects, the techniques described herein relate to a method wherein computer-executable code of one or more of the arrhythmia mapping system, the electrocardiogram system, and the path planning system execute in a trusted execution environment. In some aspects, the techniques described herein relate to a method wherein the path planning system is part of a navigation system.

In some aspects, the techniques described herein relate to one or more computing systems that provide a cardiology support system having components, the one or more computing systems including: one or more computer-readable storage mediums that store computer-executable instructions of: an electrocardiogram analytics component that inputs an electrocardiogram, generates a beat delineation and a beat classification based on the electrocardiogram, and outputs the beat delineation and the beat classification; an arrhythmia mapping component that inputs the beat delineation and the beat classification, identifies a source location of an arrhythmia and a first 3D mesh of a heart based on the beat delineation and/or the beat classification, and outputs the source location and the first 3D mesh; an image analytics component that the inputs an image, generates a second 3D mesh based on the image, and outputs the second 3D mesh; a substrate mapping component that inputs the second 3D mesh, determines cardiac wall thickness based on the second 3D mesh, and outputs an indication of the cardiac wall thickness; and a data fusion component that inputs the source location, the first 3D mesh, the second 3D mesh, and the indication of cardiac wall thickness, adjusts the source location based on difference between the first 3D mesh and the second 3D mesh, and outputs the adjusted source location, the second 3D mesh, and the indication of the cardiac wall thickness; wherein the components implement an electrophysiological procedure application programming interface that specifies behavior and parameters of push functions and behavior and parameters of pull functions so the components can exchange data during an electrophysiological procedure; and one or more processors for controlling the one or more computing systems to execute one or more of the computer-executable instructions.

All documents incorporated by reference are incorporated in their entirety for the full extent of their disclosures. In the event of inconsistencies between the language in this document and any incorporated-by-reference document, the language in the incorporated-by-reference document should be considered supplementary to that of this document and the language in this document controls.

Although the subject matter has been described in language specific to structural features and/or acts, it is to be understood that the subject matter defined in the appended claims is not necessarily limited to the specific features or acts described above. Rather, the specific features and acts described above are disclosed as example forms of implementing the claims.

I/We claim:

1. A plurality of electrophysiological procedure systems that support an electrophysiological procedure and that implement an electrophysiological procedure application programming interface, the plurality of electrophysiological procedure systems comprising:
a first electrophysiological procedure system and a second electrophysiological procedure system
wherein the first electrophysiological procedure system provides first implementations of one or more pull functions having parameters specifying data to be provided by the first electrophysiological procedure system to an invoking electrophysiological procedure system and one or more push functions having parameters specifying data being provided by an invoking electrophysiological procedure system to the first electrophysiological procedure system;
wherein the second electrophysiological procedure system provides second implementations of one or more pull functions having parameters specifying data to be provided by the second electrophysiological procedure system to an invoking electrophysiological procedure system and one or more push functions having parameters specifying data being provided by an invoking electrophysiological procedure system to the second electrophysiological procedure system;
wherein the electrophysiological procedure application programming interface specifies behavior and parameters of push functions and behavior and parameters of pull functions so that electrophysiological procedure systems that implement push functions and pull functions can exchange data during an electrophysiological procedure;
wherein the first electrophysiological procedure system includes computer-executable instructions for controlling one or more computing systems to execute the first implementations; and
wherein the second electrophysiological procedure system includes computer-executable instructions for controlling one or more computing systems to execute the second implementations.

2. The plurality of electrophysiological procedure systems of claim 1 wherein the first electrophysiological procedure system can exchange data with a third electrophysiological procedure system rather than the second electrophysiological procedure system when the third electrophysiological procedure system provides third implementations of the same pull functions and pull functions provided by the second electrophysiological procedure system.

3. The plurality of electrophysiological procedure systems of claim 1 wherein the first electrophysiological procedure system and the second electrophysiological procedure system communicate via one or more of a direct connection architecture, an intranet architecture, a shared storage architecture, an Internet architecture, and an embedded code architecture.

4. The plurality of electrophysiological procedure systems of claim 1 wherein the computer-executable instructions of the first electrophysiological procedure system are executed by a first computing system and the computer-executable instructions of the second electrophysiological procedure system execute in a trusted execution environment of the first computing system.

5. The plurality of electrophysiological procedure systems of claim 1 wherein a trusted execution environment of a computer system provides to the first electrophysiological procedure system an attestation of the computer-executable instructions of the second electrophysiological procedure system.

6. The plurality of electrophysiological procedure systems of claim 1 wherein data exchanged between the first electrophysiological procedure system and the second electrophysiological procedure system is encrypted data of a push function that is encrypted using public/private key encryption.

7. The plurality of electrophysiological procedure systems of claim 1 wherein the electrophysiological procedure application programming interface specifies a pull function and a push function for user interface input data, user interface output data, electrocardiogram data, mesh data, image data, ablation parameter data, navigation data, and electroanatomical mapping data.

8. The plurality of electrophysiological procedure systems of claim 7 wherein the electrophysiological procedure application programming interface specifies parameters for a pull function and a push function that include data type, data format, and data characteristics.

9. The plurality of electrophysiological procedure systems of claim 7 wherein the electrophysiological procedure application programming interface specifies parameters for a pull function that include a requestor and parameters for a push function that include a supplier.

10. The plurality of electrophysiological procedure systems of claim 1 wherein the first electrophysiological procedure system verifies that it is executing on a computing system that it is authorized to execute on based on decrypting data encrypted by a trusted execution environment of the computing system, the data encrypted with a private key of a public/private keypair of the trusted execution environment and decrypted with a public key of the public/private keypair.

11. The plurality of electrophysiological procedure systems of claim 1 wherein the electrophysiological procedure application programming interface specifies encryption of data to comply with the Health Insurance Portability and Accountability Act and/or the General Data Protection Regulation.

12. A method performed by one or more computing systems for executing computer-executable code of a first electrophysiological procedure system and a second electrophysiological procedure system, the method comprising:
storing first computer-executable code of the first electrophysiological procedure system in memory of the one or more computing systems, the first computer-executable code implementing one or more functions of an electrophysiological procedure application programming interface;
storing second computer-executable code of the second electrophysiological procedure system in protected memory of a trusted execution environment of the one or more computing systems, the second computer-executable code implementing one or more functions of the electrophysiological procedure application programming interface; and
under control of the first computer-executable code,
requesting from the trusted execution environment an attestation of the second computer-executable code;
receiving from the trusted execution environment the attestation;
verifying based on the attestation that the second computer-executable code is as expected; and
invoking an invoked function of the electrophysiological procedure application programming interface implemented by the second computer-executable code;
wherein the computer-executable code of the invoked function is executed within the trusted execution environment and
wherein the electrophysiological procedure application programming interface specifies behavior and parameters of push functions and behavior and parameters of pull functions so that electrophysiological procedure systems that implement push functions and pull functions can exchange data during an electrophysiological procedure.

13. The method of claim 12 wherein the computer-executable code of the first electrophysiological procedure system and the computer-executable code of the second electrophysiological procedure system execute on different computing systems.

14. A method performed by one or more computing systems during an ablation procedure for a patient, the method comprising:
under control of an arrhythmia mapping system that implements an electrophysiological procedure application programming interface,
sending to an electrocardiogram system a first request to invoke an electrocardiogram pull function of the electrophysiological procedure application programming interface to retrieve an electrocardiogram of the patient;
receiving from the electrocardiogram system a second request to invoke an electrocardiogram push function of the electrophysiological procedure application programming interface, the second request specifying an electrocardiogram of the patient;
identifying a source location of an arrhythmia based on the electrocardiogram of the patient; and
sending to a path planning system a third request to invoke an ablation parameter push function of the electrophysiological procedure application programming interface, the third request specifying the source location;
under control of the electrocardiogram system,
receiving from the arrhythmia mapping system the first request to invoke the electrocardiogram pull function;
retrieving an electrocardiogram of the patient;
sending to the arrhythmia mapping system the second request to invoke the electrocardiogram push function; and
under control of the path planning system,
receiving from the arrhythmia mapping system the third request to invoke the ablation parameter push function; and
generating catheter route for the ablation procedure based on the source location
wherein the electrophysiological procedure application programming interface specifies behavior and parameters of push functions and behavior and parameters of pull functions so that electrophysiological procedure systems that implement push functions and pull functions can exchange data during an electrophysiological procedure.

15. The method of claim 14 wherein the electrophysiological procedure application programming interface specifies encryption of data to comply with the Health Insurance Portability and Accountability Act and/or the General Data Protection Regulation.

16. The method of claim 14 wherein the arrhythmia mapping system verifies that it is executing on a computing system that it is authorized to execute on based on decrypting data encrypted by a trusted execution environment of the computing system, the data encrypted with a private key of a public/private keypair of the trusted execution environment and decrypted with a public key of the public/private keypair.

17. The method of claim 14 wherein computer-executable code of one or more of the arrhythmia mapping system, the electrocardiogram system, and the path planning system execute in a trusted execution environment.

18. The method of claim 14 wherein the path planning system is part of a navigation system.

19. One or more computing systems that provide a cardiology support system having components, the one or more computing systems comprising:
one or more computer-readable storage mediums that store computer-executable instructions of:
an electrocardiogram analytics component that inputs an electrocardiogram, generates a beat delineation and a beat classification based on the electrocardiogram, and outputs the beat delineation and the beat classification;
an arrhythmia mapping component that inputs the beat delineation and the beat classification, identifies a source location of an arrhythmia and a first 3D mesh of a heart based on the beat delineation and/or the beat classification, and outputs the source location and the first 3D mesh;
an image analytics component that the inputs an image, generates a second 3D mesh based on the image, and outputs the second 3D mesh;
a substrate mapping component that inputs the second 3D mesh, determines cardiac wall thickness based on the second 3D mesh, and outputs an indication of the cardiac wall thickness; and
a data fusion component that inputs the source location, the first 3D mesh, the second 3D mesh, and the indication of cardiac wall thickness, adjusts the source location based on difference between the first 3D mesh and the second 3D mesh, and outputs the adjusted source location, the second 3D mesh, and the indication of the cardiac wall thickness;
wherein the components implement an electrophysiological procedure application programming interface that specifies behavior and parameters of push functions and behavior and parameters of pull functions so the components can exchange data during an electrophysiological procedure;
and
one or more processors for controlling the one or more computing systems to execute one or more of the computer-executable instructions.
